(19) United States
(12) Patent Application Publication
(10) Pub. No.: US 2008/0027415 A1
(43) Pub. Date: Jan. 31, 2008

(54) VASCULAR ACCESS DEVICE VOLUME DISPLACEMENT

(75) Inventors: S. Ray Isaacson, Roy, UT (US); Austin Jason McKinnon, Herriman, UT (US); Marty L. Stout, South Weber, UT (US); Mark A. Crawford, Sandy, UT (US); Weston F. Harding, Lehi, UT (US); Sean Squire, Draper, UT (US); Bryan G. Davis, Sandy, UT (US); Christopher N. Cindrich, Draper, UT (US); Thomas M. Miner, Riverton, UT (US); Chad M. Adams, West Jordan, UT (US)

Correspondence Address: David W. Highet;Becton, Dickinson and Company, (Metcalf Intellectual Property Law, LLC), 1 Becton Drive, MC 110, Franklin Lakes, NJ, 07417-1880, US

(73) Assignee: BECTON, DICKINSON AND COMPANY, Franklin Lakes, NJ (US)

(21) Appl. No.: 11/829,007

(22) Filed: Jul. 26, 2007

Related U.S. Application Data
(60) Provisional application No. 60/820,657, filed on Jul. 28, 2006.

(51) Int. Cl. A61M 25/16

(52) U.S. Cl. 604/539

(57) ABSTRACT

A medical device may include a vascular access device with an access port which may include a septum and a slit. The slit may be formed on the inner surface of the body of the septum and the access port may be capable of receiving a separate access device through the slit of the septum. The medical device may also include a flexible member which expands to create an additional volume within the access port when the port is accessed by the access device. A method of controlling volume displacement a chamber of a medical device may include decreasing the volume of a chamber of an extravascular system by inserting a substance having a mass into the chamber and/or increasing the volume of the chamber simultaneously and commensurately with the mass of the substance inserted into the chamber.

### RELATED APPLICATIONS

This application claims the benefit of U.S. Provisional Application No. 60/820,657, filed Jul. 28, 2006, entitled VASCULAR ACCESS DEVICE VOLUME DISPLACEMENT, which is incorporated herein by reference.

### BACKGROUND OF THE INVENTION

The present disclosure relates to the displacement of volume in medical devices such as vascular access devices to provide infusion or other therapy to patients. Infusion therapy is one of the most common health care procedures. Hospitalized, home care, and other patients receive fluids, pharmaceuticals, and blood products via a vascular access device inserted into the vascular system. Infusion therapy may be used to treat an infection, provide anesthesia or analgesia, provide nutritional support, treat cancerous growths, maintain blood pressure and heart rhythm, or many other clinically significant uses.

Infusion therapy is facilitated by vascular access devices located outside the vascular system of a patient (extravascular devices). Extravascular devices that may access a patient's peripheral or central vasculature, either directly or indirectly, include closed access devices, such as the BD Q-SYTE™ closed Luer access device of Becton, Dickinson and Company; syringes; split access devices; catheters; and intravenous (IV) fluid chambers. A vascular access device may be indwelling for short term (days), moderate term (weeks), or long term (months to years). A vascular access device may be used for continuous infusion therapy or for intermittent therapy.

A common vascular access device is a plastic catheter that is inserted into a patient's vein. The catheter length may vary from a few centimeters for peripheral access to many centimeters for central access. The catheter may be inserted transcutaneously or may be surgically implanted beneath the patient's skin. The catheter, or any other extravascular device attached thereto, may have a single lumen or multiple lumens for infusion of many fluids simultaneously.

The proximal end of a vascular access device commonly includes a Luer adapter to which other medical devices may be attached. For example, an administration set may be attached to a vascular access device at one end and an IV bag at the other. The administration set is a fluid conduit for the continuous infusion of fluids and pharmaceuticals. Commonly, an IV access device is a vascular access device that may be attached to another vascular access device, closes or seals the vascular access device, and allows for intermittent infusion or injection of fluids and pharmaceuticals. An IV access device may comprise a housing and a septum for closing the system. The septum may be opened with a blunt cannula or a male Luer of a medical device.

Complications associated with infusion therapy may cause significant morbidity and even mortality. One significant complication is catheter related blood stream infection (CRBSI). An estimate of 250,000-400,000 cases of central venous catheter (CVC) associated BSIs occur annually in US hospitals. Attributable mortality is an estimated 12%-25% for each infection and a cost to the health care system of $25,000-$56,000 per episode.

Vascular access device infection resulting in CRBSIs may be caused by pathogens entering the fluid flow path from the displacement of blood subsequent to catheter insertion. Studies have shown the risk of CRBSI increases with catheter indwelling periods. This may be due, at least in part, to the displacement of blood from the vascular system of a patient to an extravascular device, such as the catheter. When contaminated, pathogens adhere to the vascular access device, colonize, and form a biofilm. The biofilm is resistant to most biocidal agents and provides a replenishing source for pathogens to enter a patient's bloodstream and cause a BSI.

Certain extravascular devices can operate with each other to form a continuous, extravascular system that provides fluid access to the vascular system, yet is entirely sealed from the external surrounding environment. Such a sealed system limits or supposedly prevents unwanted bacteria from entering from the external surrounding environment through the extravascular devices to the vascular system of a patient.

However, a sealed system of extravascular devices (extravascular system) may function as a closed or sealed vacuum, capable of drawing blood, and consequently a culture for infection, into the extravascular system. As devices are twisted off or otherwise removed from the extravascular system, the volume of the extravascular system is sometimes slightly increased. Because extravascular systems are often less elastic than a patient's vascular system, when the volume of the extravascular system is increased, the volume of a patient's vascular system is decreased under a vacuum pressure from the extravascular system. When the volume of the vascular system decreases, blood flows or is sucked from the vascular system to the extravascular system. Further, as pressure in the extravascular system decreases below the vascular pressure of a patient, either as a result of a change in volume in the extravascular system or another event, blood will flow from the vascular system to the extravascular system.

As recognized in conjunction with the present invention, even a temporary presence of blood within an extravascular system can cause future operational challenges for that extravascular system. For example, blood that clots in the end of a catheter of an extravascular system can block future fluid flow between the extravascular system and a vascular system. If drugs and other fluid substances are forced through the extravascular system, causing the blood clot to dislodge from the extravascular system, the blood clot will enter the vascular system, causing a dangerous embolism within the patient. Finally, as discussed above, even the rapid entry and exit of blood into the catheter tip of an extravascular system will leave a residue of protein, bacteria, and other pathogens on the inner wall of the catheter. This residue may become a breeding ground for bacteria to grow, and after a given period of time, will cause the formation of a harmful biofilm that is difficult to remove or bypass during extravascular system operation.

Therefore, a need exists for systems and methods that avoid or limit the displacement of blood from a patient's vascular system into an extravascular system that is connected to the patient's vascular system.

### BRIEF SUMMARY OF THE INVENTION

The present invention has been developed in response to problems and needs in the art that have not yet been fully resolved by currently available extravascular systems, devices, and methods. Thus, these developed systems, devices, and methods provide an extravascular system that may be connected to a patient's vascular system and will limit or prevent the flow or displacement of blood from the vascular system to the extravascular system.

A medical device may include a vascular access device with an access port which may include a septum and a slit. The slit may be formed on the inner surface of the body of the septum and the access port may be capable of receiving a separate access device through the slit of the septum. The medical device may also include a flexible structure such as an elastomer which expands to create an additional volume within the access port when the port is accessed by the access device. Access by a separate access device may include either fluid infusion into the access port or the insertion of a mechanical structure into the access port.

The medical device may include a peristaltic catheter for delivering a bolus of the fluid along the length of the peristaltic catheter. The medical device may also have a flexible gate or check valve through which the fluid is infused. The medical device may include a balloon housed within a chamber or may form a twisted fluid path that untwists as the device expands. The medical device may further include an air pressure chamber where the volume within the port is housed within a fluid chamber, and the volume of the fluid chamber increases as the volume of the air pressure chamber or pressure sensitive chemical chamber changes.

The medical device may include a strut in communication with the flexible structure that may expand as the strut compresses. The medical device may have a bulb that expands when the port is accessed by the device. The medical device may also form a wall of a compression balloon and expand as the compression balloon is compressed. The medical device may also include a radial compression spring, wherein the device expands as the radial compression spring moves.

A method of controlling volume displacement of a chamber of a medical device may include decreasing the volume of a chamber of an extravascular system by inserting a substance having a mass into the chamber and increasing the volume of the chamber simultaneously and commensurately with the mass of the substance inserted into the chamber. The substance may be a mechanical structure that may include a tip of a syringe. The substance may additionally or alternatively be a fluid.

A medical device may also include means for increasing the volume of a chamber in an extravascular system where the means for increasing the volume commensurately communicates with means for decreasing the volume of the chamber. The means for increasing the volume may be housed within a closed Luer access device.

These and other features and advantages of the present invention may be incorporated into certain embodiments of the invention and will become more fully apparent from the following description and appended claims, or may be learned by the practice of the invention as set forth hereinafter. The present invention does not require that all the advantageous features and all the advantages described herein be incorporated into every embodiment of the invention.

### BRIEF DESCRIPTION OF THE SEVERAL VIEWS OF THE DRAWINGS

In order that the manner in which the above-recited and other features and advantages of the invention are obtained will be readily understood, a more particular description of the invention briefly described above will be rendered by reference to specific embodiments thereof which are illustrated in the appended drawings. These drawings depict only typical embodiments of the invention and are not therefore to be considered to limit the scope of the invention.

FIG. 1 is a perspective view of an extravascular system connected to the vascular system of a patient.

FIG. 2 is a partial cross section view of an extravascular system which includes a flexible member.

FIG. 3 is a partial cross section view of an extravascular system which includes a flexible member having a bolus of fluid.

FIG. 4 is a partial cross section view of an extravascular system which has passed the bolus of fluid and returned to its original state.

FIG. 5 is a bottom perspective view of a vascular access device.

FIG. 6 is a partial cross section view of a check valve and flexible gate of the vascular access device of FIG. 5.

FIG. 7 is a partial cross section view of the check valve of FIG. 5 shown with an amount of fluid being infused.

FIG. 8 is a partial cross section view of the check valve of FIG. 5 returning to its original position.

FIG. 9 is a cross section view of a vascular access device attached to a flexible member that is a pleated or ribbed bulb or balloon.

FIG. 10 is a cross section view of the vascular access device and flexible member of FIG. 9 with fluid infused.

FIG. 11 is a plan view of a vascular access device where the flexible member forms a twisted fluid path.

FIG. 12 is a plan view of the vascular access device of FIG. 11 where the flexible member is untwisted and the fluid path shows a larger volume.

FIG. 13 is a plan view of the vascular access device of FIG. 11 where the flexible member returns to its original twisted position.

FIG. 14 is a partial cross section view of a septum having a flexible member with an air pressure chamber.

FIG. 15 is a partial cross section view of a tip inserted into the septum of FIG. 14.

FIG. 16 is a partial cross section view of a septum having a flexible member with an air pressure chamber.

FIG. 17 is a partial cross section view of a tip inserted into the septum of FIG. 16.

FIG. 18 is a partial cross section view of a vascular access device in communication with a strut.

FIG. 19 is a side view of the strut of FIG. 18 buckled.

FIG. 20 is a partial cross section view of a vascular access device having a strut attached to a knob.

FIG. 21 is a partial cross section view of a male Luer tip inserted into the vascular access of FIG. 20.

FIG. 22 is a partial cross section view of a vascular access device having a flexible member formed as a bulb attached to the floor of a septum housed within the body.

FIG. 23 is a partial cross section view of a tip inserted into the septum of FIG. 22.

FIG. 24 is a cross section view of one embodiment of the device illustrated in FIG. 23 illustrating ribs on the bulb.

FIG. 25 is a cross section view of a further embodiment of the device illustrated in FIG. 23 illustrating a pleaded configuration.

FIG. 26 is a partial cross section view of a vascular access device having a bulb with a sigmoid arm secured to the floor of a septum.

FIG. 27 is a partial cross section view of a vascular access device having a bulb with a flattened arm secured to the floor of a septum.

FIG. 28 is a perspective view of a vascular access device having a septum, a fluid path hole, a clamping ring, and a wedge.

FIG. 29 is a cross section view of the septum of FIG. 28.

FIG. 30 is a cross section view of a septum of FIG. 26 taken at a 90° angle from the cross section view of FIG. 29.

FIG. 31 is a quarter section view of the septum of FIG. 30.

FIG. 32 is a cross section view of a vascular access device having a compression balloon.

FIG. 33 is a cross section view of a vascular access device having a chamber beneath the floor of a septum filled with a substance.

FIG. 34 is a cross section view of the vascular access device of FIG. 33 shown with the male tip inserted into the septum.

FIG. 35 is a cross section view of a vascular access device showing the device prior to tip insertion.

FIG. 36 is a cross section view of the vascular access device of FIG. 35 during tip insertion.

FIG. 37 is a partial cross section view of a vascular access device having a flexible member with a ramp.

FIG. 38 is a partial cross section view of a tip inserted into the vascular access device of FIG. 37.

### DETAILED DESCRIPTION OF THE INVENTION

The presently preferred embodiments of the present invention will be best understood by reference to the drawings, wherein like reference numbers indicate identical or functionally similar elements. It will be readily understood that the components of the present invention, as generally described and illustrated in the figures herein, could be arranged and designed in a wide variety of different configurations. Thus, the following more detailed description, as represented in the figures, is not intended to limit the scope of the invention as claimed, but is merely representative of presently preferred embodiments of the invention.

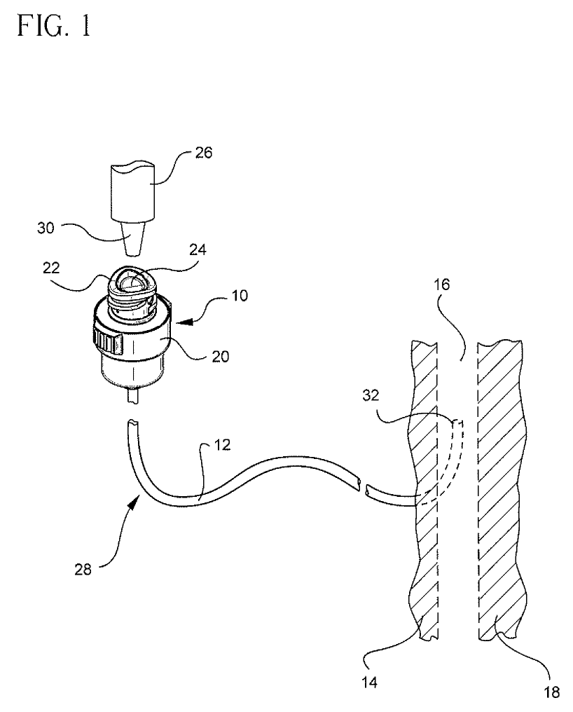

Referring now to FIG. 1, a vascular access device (also referred to as an extravascular device, intravenous access device, and/or access port) 10 is used to introduce a substance via a catheter 12 across the skin 14 and into a blood vessel 16 of a patient 18. The vascular access device 10 includes a body 20 with a lumen and a septum 22 placed within the lumen. The septum 22 has a slit 24 through which a separate extravascular device 26, such as a syringe, may introduce a substance into the vascular access device 10.

The device 10 also includes a flexible member, which may include but is not limited to an elastomer, (discussed with reference to the figures below) capable of creating a volume within the vascular access device 10 and/or the extravascular system 28 to which the vascular access device 10 is connected. The elastomer, or other flexible member, capable of creating this volume creates the volume when a tip 30 of the separate device 26 is inserted into the vascular access device 10 though the slit 24 of the septum 22. Normally, when the tip 30 is inserted into the device 10, the volume of the extravascular system 28 is decreased, causing fluid to flow from the system 28 into the blood vessel 16. Conversely, under normal conditions, when the tip 30 is removed from the device 10, the volume of the extravascular system 28 is increased, causing blood to flow from the blood vessel 16 into the system 28 by entering through the end 32 of the catheter 12.

As mentioned throughout this description, even a temporary presence of blood within an extravascular system 28 can cause future operational challenges for the extravascular system 28. These problems may include blood clots, fluid flow barriers, embolisms, and the production of harmful biofilm. Thus, the devices disclosed herein are provided to avoid reflux or displacement of blood from the blood vessel 16 into the catheter 12. The devices may include a flexible member capable of creating a volume when the separate device 26 is inserted into the vascular access device 10 and will permit the created volume to decrease to its original size. When the volume decreases to its original size, the decrease in volume will offset any volume displaced such that upon removal of the separate access device 26, fluid is forced distally from the vascular access device 10 or other medical device toward the vascular system of a patient. This further avoids creation of a vacuum that would cause blood to flow or be sucked from the blood vessel 16 into the catheter 12.

The vascular access device 10 may be accessed at its access port by any separate access device. Such access may include either fluid infusion into the port or the insertion of a mechanical structure such as tip 30 into the port. Many of the following several embodiments relate primarily, but not exclusively, to fluid infusion into the port.

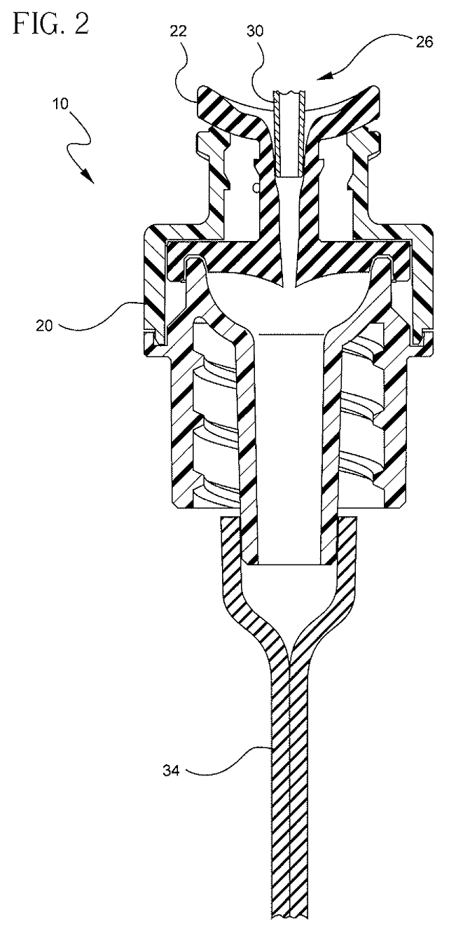

Referring now to FIG. 2, a vascular access device 10 includes at least one elastomer 34 attached to the body 20 of the vascular access device 10. The elastomer 34 is a peristaltic catheter for delivering a bolus of fluid along the length of the peristaltic catheter. During operation, a tip 30 of a separate device 26 is inserted into the access port of device 10 and fluid is delivered from the tip 30 through the lumen of the device 10 and into the lumen of the elastomer 34.

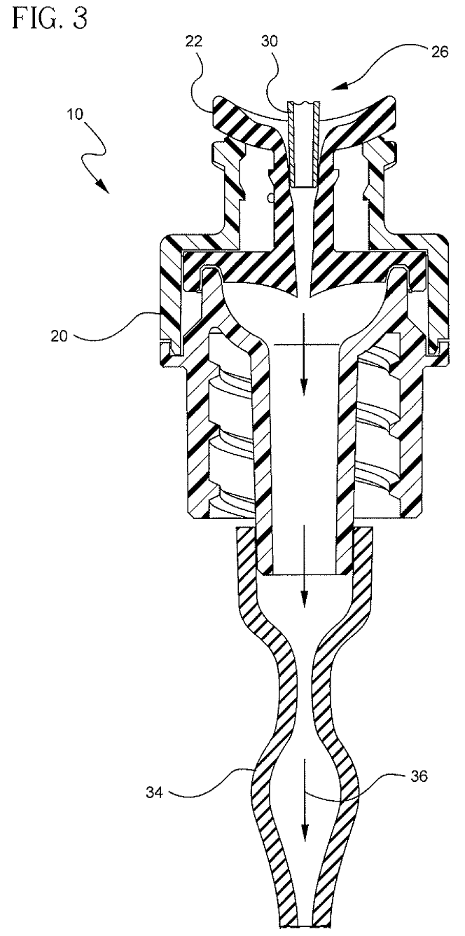

Referring now to FIG. 3, after the elastomer 34 of FIG. 2 receives a bolus of fluid, the outer walls of the elastomer 34 enclose behind the bolus of fluid, forcing the bolus of fluid in a direction 36 towards the vascular system of a patient.

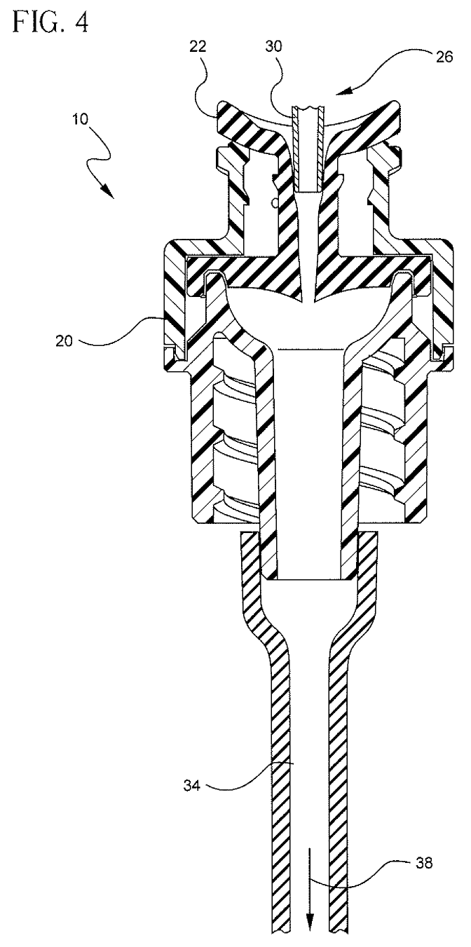

Referring now to FIG. 4, the elastomer 34 of FIGS. 2 and 3 has successfully delivered a bolus of fluid to a patient 38. The walls of the elastomer 34 have collapsed to their original resting position and are prepared to receive another bolus of fluid through the lumen of the vascular access device 10.

The embodiments shown in FIGS. 2 through 4 thus illustrate an elastomer 34 that is capable of receiving and transferring a volume of fluid in a direction 36 towards a patient in a manner that avoids any reflux of blood from the patient's vascular system into the elastomer 34 or any downstream catheter attached thereto. Thus, using the embodiment of FIGS. 2 through 4, an operator may safely remove the tip 30 of a separate device 26 from the vascular access device 10 without pulling a volume of blood from a patient 38 into any component of an extravascular system 28.

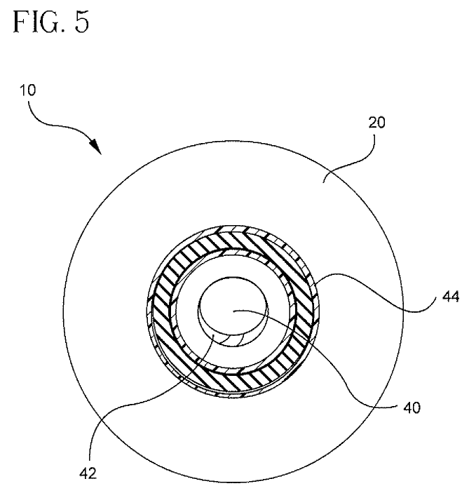

Referring now to FIG. 5, a bottom perspective view of a vascular access device 10 shows a check valve 40 capable of receiving an amount of fluid through its lumen 42. The device 10 also includes an elastomer 34 formed as a radial, flexible, elastomeric gate 44. The elastomeric gate 44 is capable of expanding to create additional volume within a fluid path that is downstream from the vascular access device 10. After fluid travels through the lumen 42 of the check valve 40, the check valve 40 closes and the pressure created by rapid infusion of the fluid causes the elastomeric gate 44 to expand inward towards the inner chamber of the vascular access device 10.

Referring now to FIG. 6, a cross section of the check valve 40 and elastomeric gate 44 of the vascular access device 10 of FIG. 5 is shown. In a resting position with no fluid infused through the check valve 40, the check valve 40 is in a straight, flush position against the body 20 of the vascular access device 10. Similarly, the elastomeric gate 44 is in a straight, resting position, since no fluid has been infused. The pressure downstream of the vascular access device 10 has thus not increased.

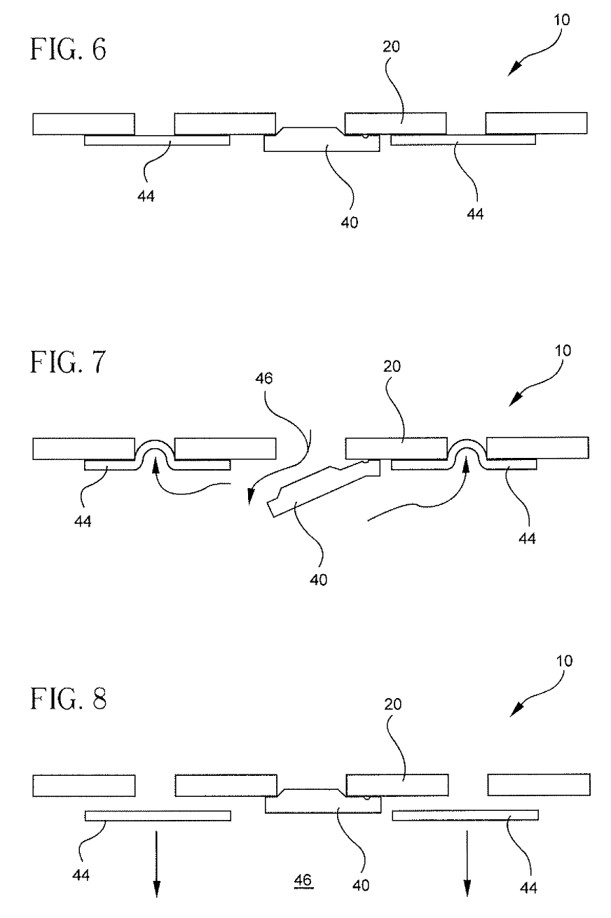

Referring now to FIG. 7, the cross section shown in FIG. 6 is shown with an amount of fluid 46 being infused through the check valve 40. As the fluid 46 is infused through the check valve 40, the elastomeric gate 44 expands under pressure into an inner chamber of the device 10. By expanding into the device 10, the elastomeric gate 44 creates an additional amount of volume in a chamber that is downstream, or outside of, the vascular access device 10. After the fluid 46 is fully infused across the check valve 40, the check valve 40 closes and the elastomeric gate 44 returns to its original staring position shown in FIG. 6. As the elastomeric gate 44 returns to its original position, it forces fluid through the downstream chamber of the extravascular system 28 and into the vascular system of a patient.

Referring now to FIG. 8, after the fluid 46 is fully infused, as mentioned above, the check valve 40 is closed and the elastomeric gate 44 returns to its original position in a direction 48 that is downstream and towards the vascular system of a patient.

In the embodiment shown in FIGS. 5 through 8, the elastic constant of the elastomeric gate 44 should be at a level that is between the blood pressure of the vascular system of a patient and the fill pressure caused when the fluid 46 is infused through the lumen 42. Thus, the elastomeric gate 44 will flex as fluid 46 is infused through the lumen 42. After the check valve 40 has closed, the elastic strength of the elastomeric gate 44 will be strong enough to return to its original position as shown in FIGS. 6 and 8. The fluid 46 will then continue along its path towards the vascular system of a patient.

Thus, the embodiment of FIGS. 5 through 8 permits an operator to insert a separate device into the vascular access device 10 and infuse a fluid 46. After the fluid 46 is fully infused, the separate device may be removed from the vascular access device 10 without pulling any blood from the vascular system of a patient into the extravascular system 28 to which the vascular access device 10 is attached.

The general concepts and various elements and configurations of the embodiment of FIGS. 5 through 8 may be modified significantly in order to achieve the principals illustrated therein and will still come within the scope of the present invention. For example, the elastomer of FIGS. 5 through 8 may be modified and placed at any point along, or adjacent to, the path of an extravascular system. Further, the elastomer 34 may include any number of radial, linear, or other flexible gates that are capable of expanding when a fluid is infused and contracting when one or more valves are closed.

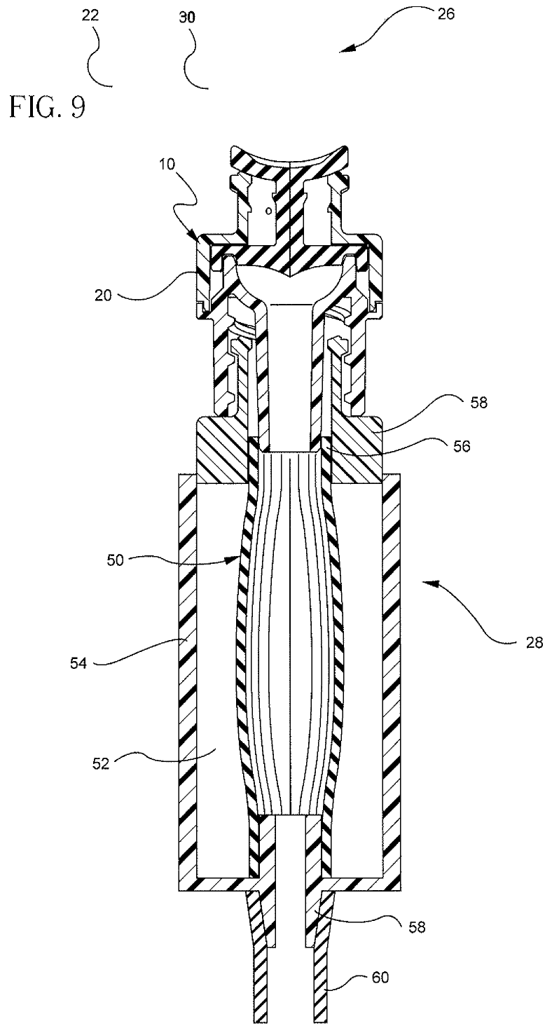

Referring now to FIG. 9, a vascular access device 10 is attached to a flexible member that is a pleated or ribbed bulb or balloon 50. The balloon 50 resides within a chamber 52 of a housing 54. The housing 54 includes female threads 56 to be attached to the male threads 58 of the vascular access device 10 at a first end. The housing 54 also includes a male connector 58 at a second end to which a catheter 60 or other downstream device of an extravascular system 28 may be attached.

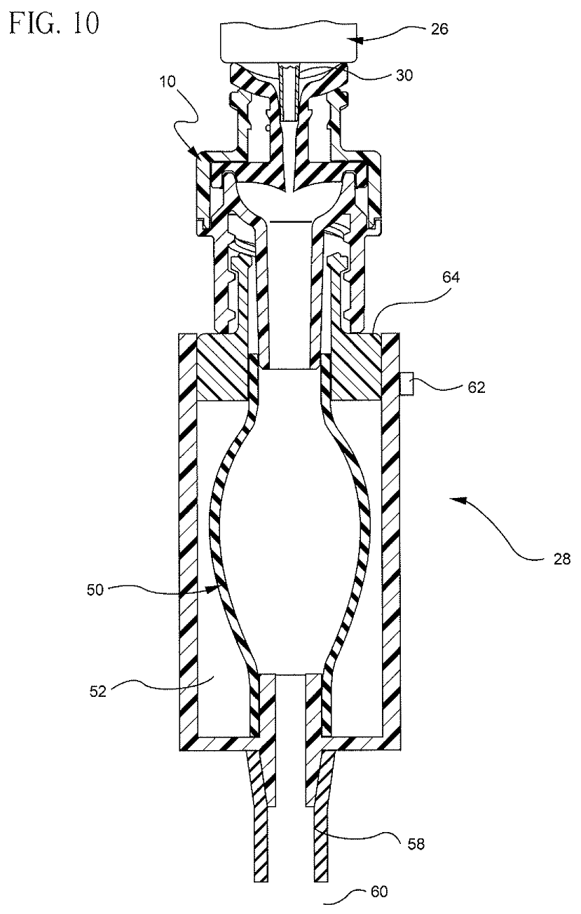

Referring now to FIG. 10, the extravascular system 28 of FIG. 9 is shown with the male tip 30 of a separate device 26 attached to the vascular access device 10. Fluid is infused from the separate device 26 through the device 10 into the balloon 50 causing the balloon 50 to expand under pressure caused from the infused fluid. The balloon 50 expands either because the male connector 58 includes a closed valve that inhibits fluid flow, or because the speed at which the fluid is infused through the vascular access device 10 into the balloon 50 is greater than the speed at which the fluid escapes the balloon 50 into the catheter 60.

A closed valve within the male connector 58 may continue to inhibit fluid flow from the balloon 50 into the catheter 60 until a user presses a button 62. The user may press the button 62 after having administered the drug or other fluid from the separate device 26 into the balloon 50. After the balloon is filled with fluid and before the user presses the button 62, the fluid may then be trapped between the closed slit septum of the vascular access device 10, or another similar valve or closure, and the valve of the male connector 58. When a user or operator presses the button 62, the valve within the male connector 58 opens releasing the pressurized fluid from the balloon 50 into the catheter 60 towards a patient.

Thus, the embodiment of FIGS. 9 and 10 provides a system which includes an elastomer that is a balloon 50 that provides pressure capable of delivering a fluid to a patient while the separate device 26 may be removed without any risk of blood flowing from the vascular system of a patient into the catheter 60 or other device of the extravascular system 28. The button 62 may be replaced by a one-way valve placed either within the vascular access device 10 or within a neck 64 of the housing 54. When fluid travels past the valve in the neck 64 and into the balloon 50, the balloon 50 is expanded and the pressure of the balloon forces the valve in the neck 64 to close after the fluid has been fully infused by the separate device 26. When the valve in the neck 64 closes and the valve in the male connector 58 is at least partially open, the fluid within the balloon 50 will be forced by the balloon 50 downstream. The fluid will travel under pressure through the male connector 58, the catheter 60, and into the vascular system of a patient. The pressurized fluid flow will not permit the fluid to flow from the vascular system of a patient into the extravascular system 28.

Referring now to FIG. 11, a vascular access device 10 may include an elastomer that forms a twisted fluid path 66 that has been manufactured to form a twisted chamber in its resting state. The twisted chamber holds a smaller volume than the chamber would hold if the elastomer were untwisted. The twisted fluid path or twisted chamber 66 may then untwist forming a chamber with a larger volume after the fluid path 66 is untwisted, causing the elastomer to expand. The twisted fluid path 66 of FIG. 11 may form any portion along or adjacent to the fluid path of an extravascular system 28.

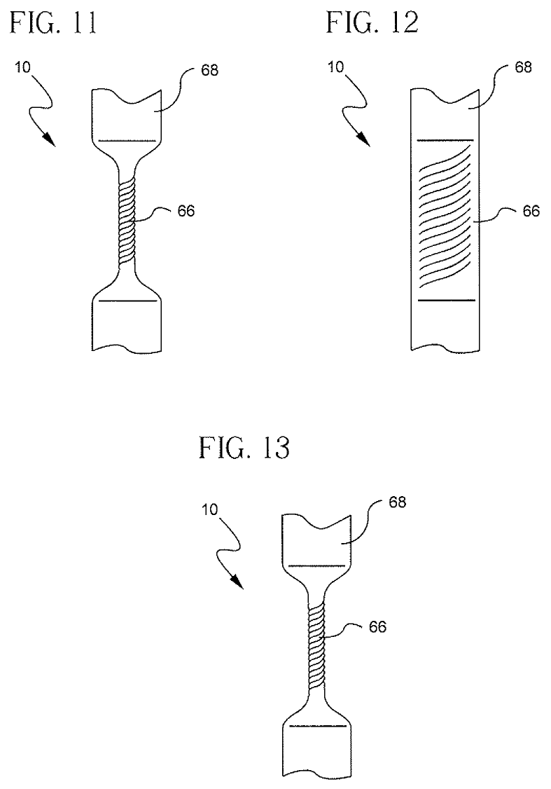

Referring now to FIG. 12, after the twisted fluid path 66 is untwisted, the cross section of the fluid path shows a larger volume than the cross section of twisted fluid path 66 of FIG. 11. The twisted fluid path 66 is untwisted upon the initiation of any twisting or other similar action or articulation that would cause the elastomer to untwist. For example, in a closed Luer access device that is a vascular access device 10, the device 10 includes male threads that are twisted onto the male tip of a syringe. When the device 10 is attached to a syringe, the two devices are twisted together.

During the twisting action required to attach the two devices together, the twisted fluid path 66 will move from an original, resting, twisted position shown in FIG. 11, to an untwisted position of larger volume shown in FIG. 12. Such action can occur when the twisted fluid path 66 is attached at a first end 68 to the separate device 26 such as a syringe. The syringe will initially combine with or otherwise secure the first end 68 of the twisted fluid path 66 and, while the male Luer of the syringe is twisted, the first end 68 will twist with the male Luer causing the twisted fluid path 66 to open and untwist. Subsequently, as the male Luer of a separate device 26 is removed, the first end 68 will untwist, causing the twisted fluid path 66 to return to its original position shown in FIG. 11.

Referring now to FIG. 13, the twisted fluid path 66 is shown returned to its original resting position of a lesser volume after a syringe or other separate device 26 has been removed from the vascular access device 10.

The embodiment of the elastomer forming a twisted fluid path 66 shown in FIG. 11 through 13 thus illustrates an elastomer that is capable of providing an additional amount of volume within the fluid path of an extravascular system 28 when the male Luer of a separate device 26 is attached to the extravascular system 28. The male Luer or tip 30 of the separate device 26 takes up volume within the extravascular system which is simultaneously and commensurately offset with the created volume shown by the untwisted fluid path 66 of FIG. 12. The offset volume of the untwisted fluid path 66 of FIG. 12 thus decreases the likelihood of, or eliminates, the risk that blood would travel from the vascular system of a patient into the extravascular system 28 to which the twisted fluid path 66 is attached upon insertion and/or retraction of the male tip 30 of a separate device 26.

In an alternate embodiment, the twisted fluid path 66 of FIGS. 11 through 13 may untwist under the pressure of a fluid received from a separate device 26. Thus in this particular embodiment, the attachment of a separate device 26 to the device 10 need not be the means by which the twisted fluid path 66 is untwisted. Rather, the pressure of the fluid sent from the separate device 26 as it travels through the twisted fluid path 66 will force the twisted fluid path to untwist creating a larger volume and then to resume into its original twisted position after fluid flow pressure decreases. This embodiment of fluid pressure used to open the twisted fluid path 66 may be used in combination with the embodiment of a separate device 26 used to untwist the first end 68 of the twisted fluid path 66.

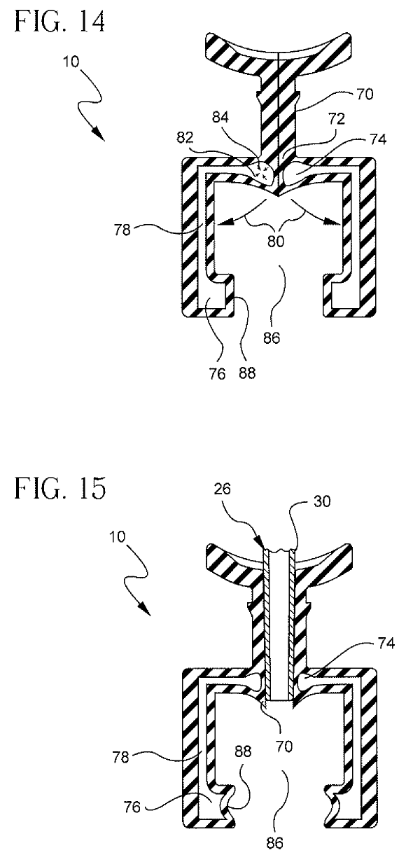

Referring now to FIG. 14, a vascular access device 10 includes a septum 70 made of an elastomer 72. Within the body of the elastomer 72, an air pressure chamber 74 communicates with a second air chamber 76 through an air pressure channel 78. As the male tip 30 of a separate device 26 is inserted into the slit of the septum 70, the septum moves in a downward and outward direction 80 causing the air pressure chamber 74 to compress in a direction 82 and simultaneously expand in a direction 84, yielding a net increase in volume within the air pressure chamber 74. When the air pressure chamber 74 increases in volume, air travels from the second air chamber 76 through the air pressure channel 78 into the air pressure chamber 74 causing the second air chamber 76 to collapse. When the second air chamber 76 collapses, the volume of a fluid chamber 86 within the vascular access device increases to offset the decrease in volume caused by the insertion of the tip 30.

Referring now to FIG. 15, the vascular access device 10 of FIG. 14 is shown with the tip 30 of a separate device 26 inserted in the septum 70. As previously described, the insertion of the tip 30 causes the air pressure chamber 74 to increase in volume and the second air chamber 76 to commensurately decrease in volume. The second air pressure chamber 76 decreases in volume because the sidewall 88 of the second air pressure chamber is thinner, or otherwise more flexible, than the remaining surrounding structures of the elastomer 72 surrounding the continuous air chamber that includes air chamber 74, channel 78, and secondary chamber 76.

The embodiment of FIGS. 14 and 15 thus shows an air pressure chamber wherein the volume of the access port of the vascular access device 10 is housed within a fluid chamber 86, and the volume of the fluid chamber 86 is capable of increasing simultaneously and commensurately with an increase in air pressure of an air pressure chamber 74. This change in volume within the interior chamber 86 offsets any increase in volume caused when the tip 30 is inserted into the vascular access device. Similarly, as the tip 30 is removed from the vascular access device, the septum 70 returns to its original position shown in FIG. 14, causing the air pressure chambers 74 and 76 to return to their original volumes, which in turn causes the interior chamber 86 to return to its original volume. The equalization of fluid and air pressure chambers of the embodiment of FIGS. 14 and 15 permits the tip 30 to be inserted into the device 10 without causing any blood to flow from the vascular system of a patient into the extravascular system 28 to which the device 10 and separate device 26 are attached.

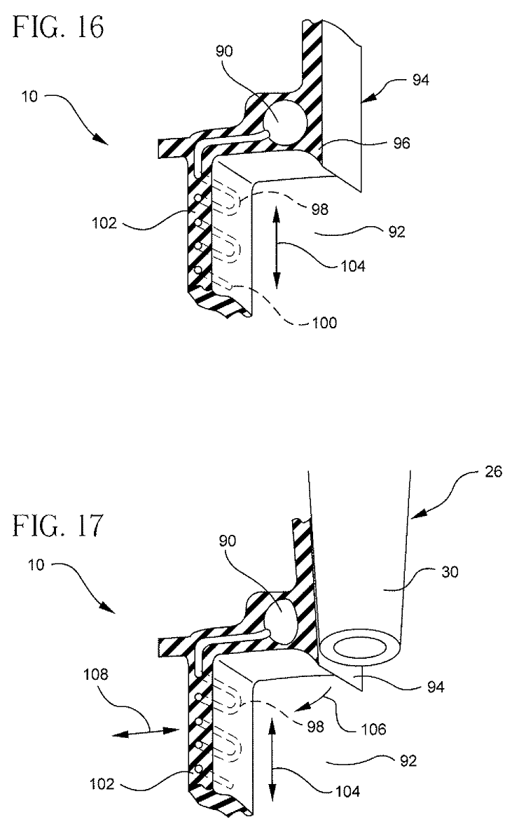

Referring now to FIG. 16, a similar embodiment to that shown in FIGS. 14 and 15 is shown wherein an air pressure chamber 90 is able to modify the volume of an interior chamber 92 as the volume of the air pressure chamber 90 changes. However, in contrast to the embodiment described in FIGS. 14 and 15, the air pressure chamber 90 increases the volume of the interior chamber 92 as the volume of the air pressure chamber 90 is decreased, rather than increased. As shown in FIG. 16, a vascular access device 10 includes a septum 94 and elastomer 96 forming the body of the septum 94. Within the housing or body of the elastomer 96, an air pressure chamber 90 is continuously attached to a serpentine air pressure channel 98. An end 100 of the serpentine air pressure channel 98 terminates the serpentine path of the air pressure channel 98 along a thin wall 102 of the elastomer 96. As the tip 30 of a separate device 26 is inserted into the septum 94 of the device 10, the elastomer 96 flexes, causing the air pressure chamber 90 to compress and reduce to a smaller volume as air is forced out of the air pressure chamber 90 into the serpentine channel 98. As air is forced through the serpentine channel 98 to the end 100, the air pressure exerted against the sidewalls of the channel 98 cause the thin wall 102 to expand in a direction 104. As the thin wall 102 expands in a direction 104, the volume of the interior chamber 92 is increased.

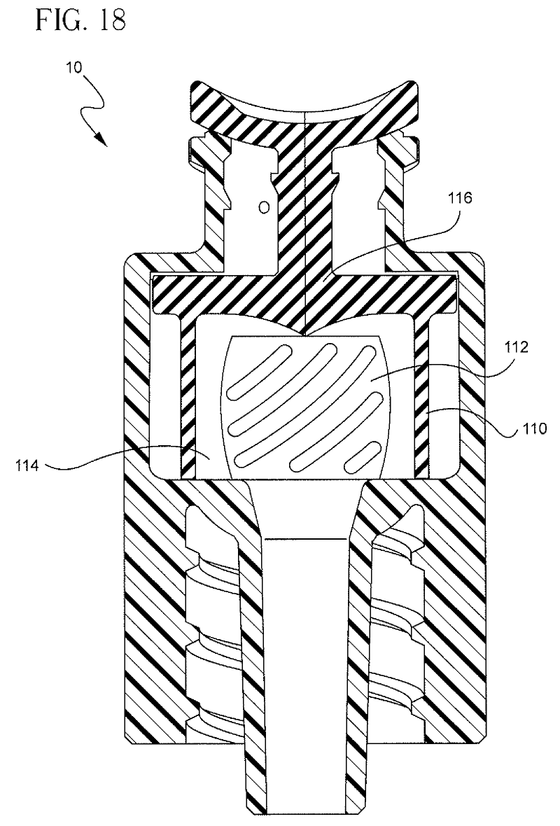

Referring now to FIG. 17, the vascular access device 10 of FIG. 16 is shown with the tip 30 of a separate device 26 inserted into the septum 94. The septum 94 is forced in a direction 106 causing the air pressure chamber 90 to contract. The contracted air pressure chamber 90 has forced air into the serpentine channel 98 causing the channel 98 and the thin sidewall 102 of the elastomer 96 to expand in a direction 104. The thin sidewall 102 is able to expand in a direction 104 in the present embodiment because the properties of the elastomer 96 along the thin sidewall 102 are such that the elastomer will expand in an axial direction 104 more easily than the elastomer 96 will expand in a lateral direction 108. The expansion of the thin sidewall 102 has created an increased amount of volume within the interior chamber 92 which has offset the decreased volume caused by the insertion of the tip 30 and the actuation of the septum 94 into the volume of the chamber 92.

Thus, similar to the embodiments of FIGS. 14 and 15, but using an opposite mechanism, the embodiment of FIGS. 16 and 17 provides an elastomer and air pressure chamber capable of displacing volume within a vascular access device in a manner that eliminates or the decreases the likelihood that blood will be drawn or will otherwise flow from the vascular system of a patient into the extravascular system 28 to which the device 10 is attached. The embodiments of FIGS. 14 through 17 are not intended to be exhaustive, rather they merely represent two examples illustrating the principals of the present invention that a change in a volume of air or fluid can result in a change in volume of an interior chamber consistent with the objectives of the present invention.

Referring now to FIG. 18, a vascular access device 10 includes an elastomer 110 that is in communication with a strut 112, or series of struts. The strut 112 is placed against the inner surface of the wall of the elastomer 110 within an interior chamber 114. When the tip 30 of a separate device 26 is inserted into the septum 116 of the device 10, axial pressure caused by the opening of the septum 116, and the downward force of the tip 30, causes the strut or series of struts 112 to buckle in an outward direction against the inner wall of the elastomer 110, which in turn causes the elastomer 110 to expand in an outward direction. As the strut 112 and elastomer 110 expand and buckle in an outward direction, the internal volume on the interior chamber 114 increases.

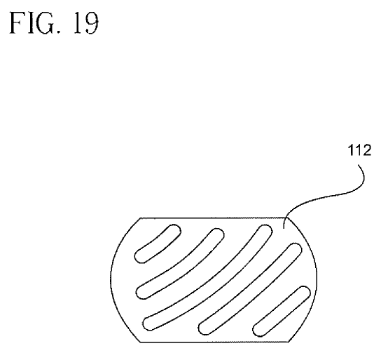

Referring now to FIG. 19, the strut 112 of the device 10 of FIG. 18 is shown. The strut 112 is shown buckled and extended outward under the axial pressure of an opening septum 116 and inserted tip 30. The compressed strut can be a single continuous piece capable of expanding and bulging outwards under axial compression, or the strut 112 may be a series of multiple joints, struts, or other members that work in combination to achieve a similar mechanism.

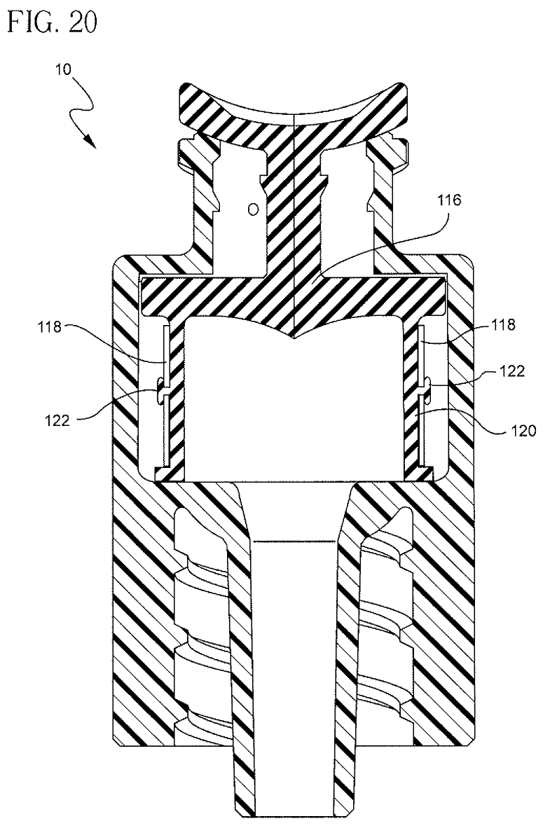

Referring now to FIG. 20, a vascular access device 10 includes a strut 118 on the external surface of an elastomer 120. The strut 118 is attached to a knob 122 of the elastomer 120 in a manner that causes the strut 118 and elastomer 120 to move in concert with each other.

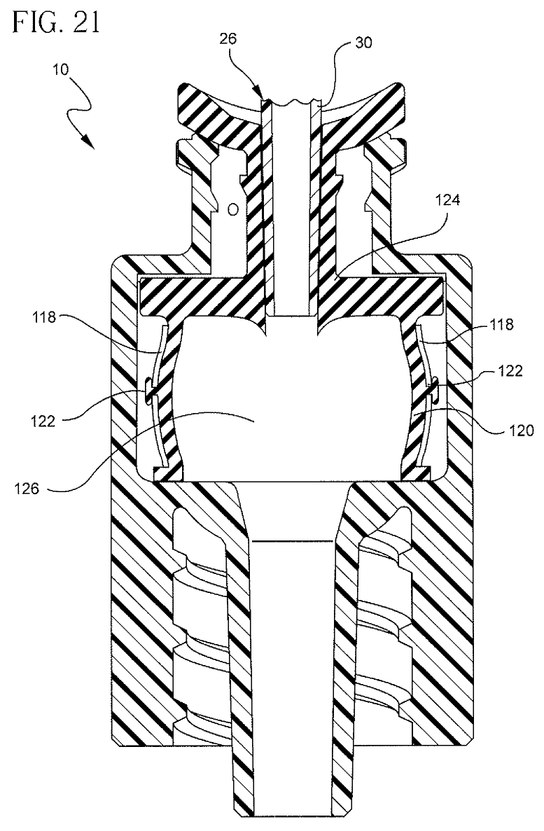

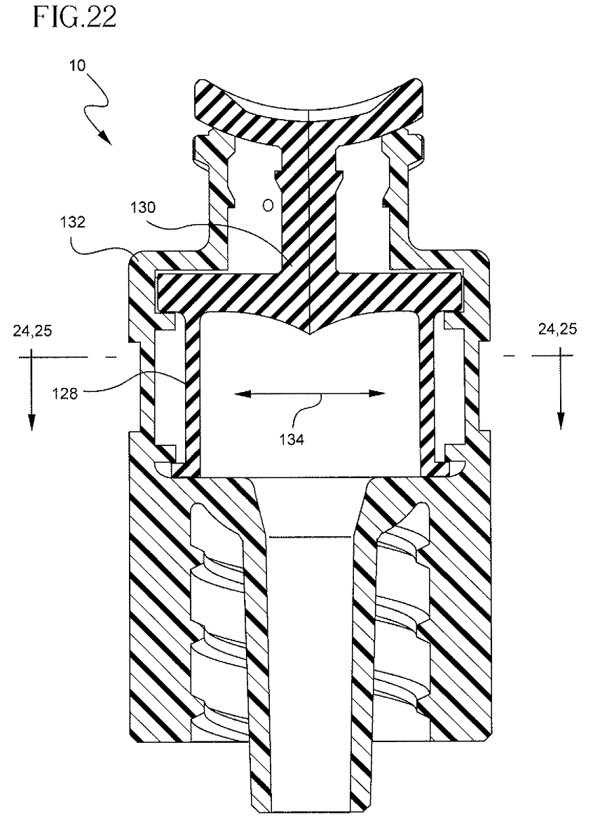

Referring now to FIG. 21, the vascular access device 10 of FIG. 20 is shown with the male Luer tip 30 of a separate device 26 inserted into the septum 124 of the device 10. The downward force of the tip 30 and the opening of the septum 124 cause downward axial compression upon the strut 118 and its attached elastomer 120, forcing the strut 118 and the elastomer 120 to expand, or bulge, outward. As the strut 118 and the elastomer 120 bulge outward, the internal volume of an interior chamber 126 within the device 10 increases. The increase in volume of the interior chamber 126 offsets any decrease in volume caused by the opening of the septum 124 and/or the insertion of the tip 30. The offset volume permits the vascular access device 10 to receive any portion of a separate device 26 within its septum 124 without causing or creating an environment where blood will travel from a vascular system of a patient into the extravascular system 28 when the inserted portion of the separate device 26 is removed from the device 10. An elastomer of the present invention need not work in combination with a buckling structure such as the struts illustrated with reference to FIGS. 18 through 21, as shown in the following embodiments.

Referring now to FIG. 22, a vascular access device 10 includes an elastomer formed as a bulb 128 attached to the floor of a septum 130 housed within the body 132 of the device 10. In its resting state, the bulb 128 is biased, oriented, mechanically structured, or otherwise configured to move in a direction 134 when the septum 130 is actuated.

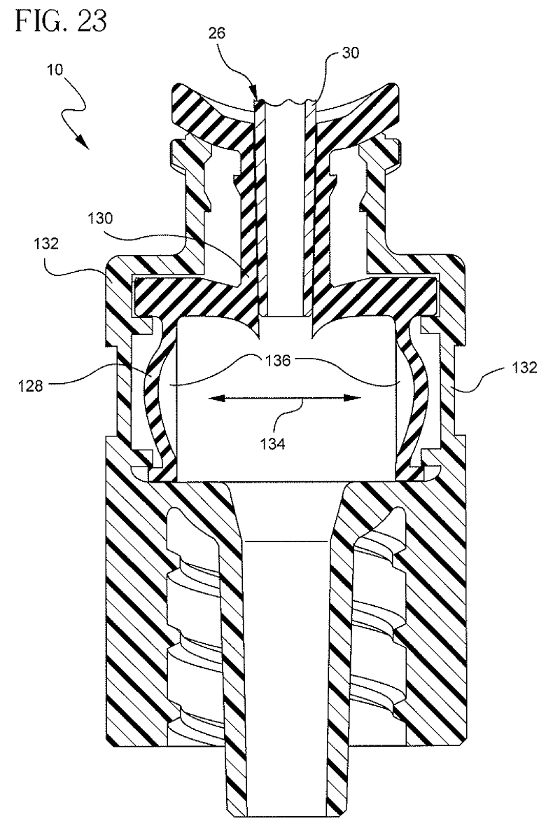

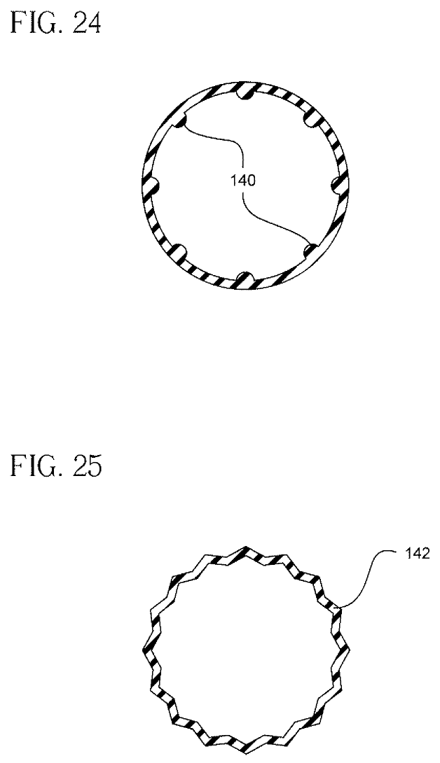

As shown in FIG. 23, the vascular access device 10 of FIG. 22 is shown with the male tip 30 of a separate device 26 inserted into the septum 130, causing the bulb 128 to travel in a direction 134. As the bulb 128 travels in a direction 134, towards the internal surface of the body 132 of the device 10, an additional amount of storage volume 136 is created within the interior chamber 138 of the device 10. The bulb 128 opens and expands naturally under a trampoline effect as the septum 130 opens under pressure from the tip 30. As illustrated in FIGS. 24 and 25, ribs 140 and/or pleats 142 may be added with other similar mechanical structures in combination or separately to the internal and/or external surface of the bulb 128 to create the mechanical properties required to permit the bulb 128 to travel in a direction 134 when influenced by an actuated septum 130. The ribs 140 and pleats 142 are shown in cross section view of two potential embodiments of the bulb 128 as shown in the additional drawings of FIG. 23. Since the embodiment of FIGS. 22 and 23 may force the septum 130 to remain open while an amount of fluid pressure within the interior chamber 138 is placed upon the bulb 128 in a direction 134, a separate embodiment providing structure that permits the septum 130 to close in the presence of such pressure may be preferred as described with reference to FIG. 24.

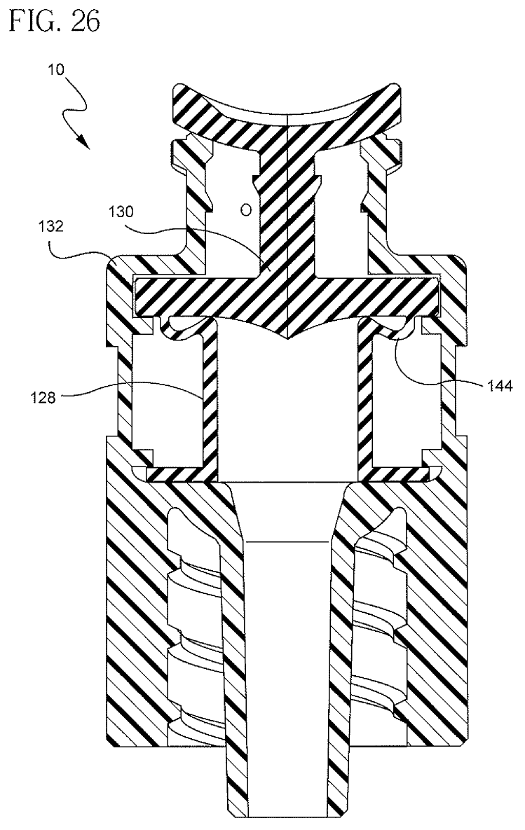

Referring now to FIG. 26, an alternate embodiment of the embodiment shown in FIGS. 22 and 23 includes a bulb 128 with a sigmoid arm 144 attached to the upper portion of the bulb 128. The sigmoid arm 144 secures the bulb 128 to the floor of the septum 130. The sigmoid arm 144 permits the bulb 128 to bulge under fluid back pressure while allowing the duckbill portion of the septum 130 to close.

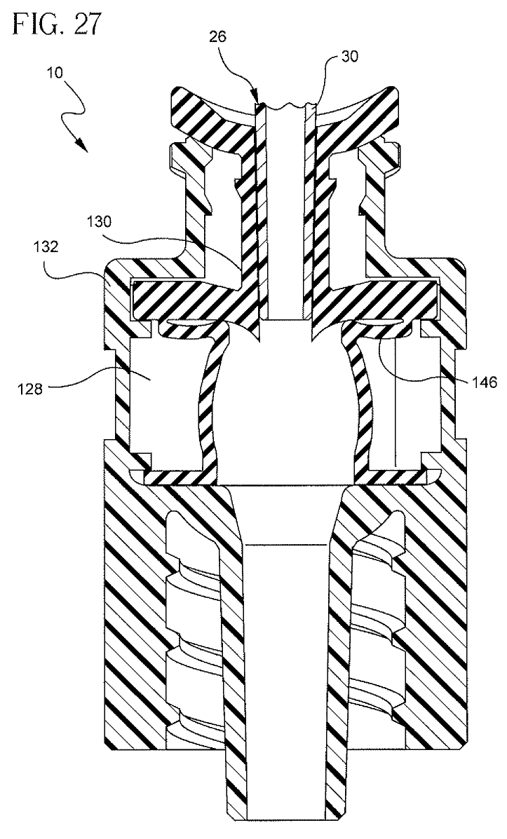

Referring now to FIG. 27, the sigmoid arm 144 of the embodiment of FIG. 24 may be flattened to form flattened arm 146 on the upper portion of the bulb 128. The flattened arm 146 may reside adjacent to or may be in direct contact with the floor of the septum 130 in order to pinch shut in a manner that keeps fluid, proteins, bacteria or other pathogens from growing and residing within the chamber 148. The arm 146 may be flattened such that the chamber 148 may be entirely eliminated during use and actuation of the bulb 128 and the septum 130.

Thus, the embodiments of FIGS. 26 and 27 provide the additional mechanical structure necessary to permit the bulb to remain open under the pressure of a fluid within an interior chamber 138 (as shown in FIG. 23), while permitting the septum 130 to close. The additional structure, such as sigmoid arm 144 and flattened arm 146 provide a back pressure release that will permit the septum 130 to close and the tip 30 of a separate device 26 to be removed while the commensurate volume associated with the closure of the septum 130 and removal of the tip 30 is increased within the interior chamber 138. The interior chamber 138 maintains an increased volume while the bulb 128 is still under pressure. The volume of the interior chamber 138 then decreases and empties, forcing fluid from the interior chamber 138 downstream through the remainder of the extravascular system 128 and into the vascular system of a patient. Thus, the back pressure failure release, or counter measure, of the embodiments of FIGS. 26 and 27 permits an operator to remove a separate device 26 from a vascular access device 10 without any risk of the blood of the vascular system of a patient entering into the extravascular system 28 during operator use.

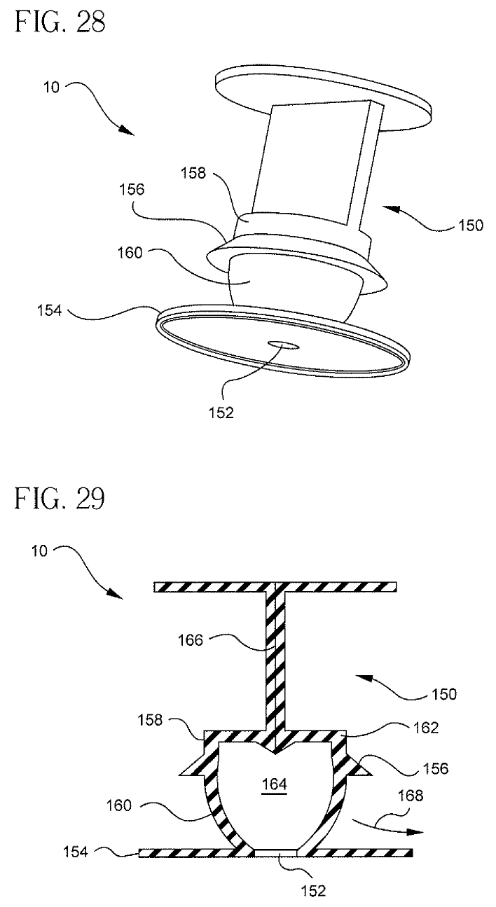

Referring now to FIG. 28, a vascular access device 10 includes a septum 150 having a fluid path hole 152, a clamping ring 154, and a wedge 156. The fluid path hole 152 should be aligned with the hole or lumen of a device in series with the septum 150. And, the hole or lumen that is in communication with the fluid path hole 152 should be slightly smaller or the same diameter as the fluid path hole 152 in order to avoid fluid entrapment outside of the fluid path below the fluid path hole 152. Fluid entrapment is any space where fluid may reside outside the direct fluid path where the fluid must travel. Fluid entrapment permits the creation of eddy currents and other stagnant fluid that may reside within the vascular access device 10 for a period of time and later mix with fluid that is administered to a patient. When the stagnant fluid is later mixed with fluid that is administered to a patient, the mixture may yield unpredictable or unsafe results for the patient. The design of the wedge 156 is structured in order to guide the septum 150 into its original, resting, unactuated position. An upper straight wall 158 is formed on the outer surface of the septum 150 in order to prevent the internal opening of the septum 150 from opening when the fluid path within bulb 160 is pressurized.

Referring now to FIG. 29, the septum 150 of FIG. 28 is shown in cross section view. As shown, the straight wall 158 is configured to support and close the floor 162 of the septum 150 when pressurized fluid is contained within the inner chamber 164 of the bulb 160. The straight wall 158 thus acts as a vertical containment wall, preventing fluid from escaping from the chamber 164 through the slit 166 of the septum 150. This design of an elastomeric septum 150 provides a vascular access device 10 that allows for better fluid flushing, less volume within chamber 164 required to prime the fluid path of the device 10, and potential volume displacement when the tip 30 of a separate device 26 is removed from the slit 166.

When a male Luer or tip 30 is inserted into the slit 166 of the septum 150, the floor 162 of the septum 150 extends into the chamber 164 allowing the floor 162 to open outward. As the tip 30 is inserted into the slit 166, the septum 150 is forced downward under axial pressure causing thin sidewalls of the bulb 160 to buckle and bend outwards in a direction 168. When the sidewalls of the bulb 160 bend outwards, the volume of the chamber 164 is increased to offset the decrease in volume caused by entry of the floor 162 into and towards the chamber 164. Fluid is then injected through the tip 30 and the separate device 26 is removed.

As the separate device 26 is removed, the thin sidewalls of the bulb 160 return to their original positions, expelling fluid from the chamber 164 and forcing the lower part or floor 162 of the septum 150 back into its original position with the straight vertical containment wall 158 compressed against the floor 162 to keep the floor 162 from opening. The bulb 160 includes thin sidewalls that are curved in a direction away from the chamber 164 and are pleated or otherwise mechanically altered to promote buckling away from the fluid path hole 152 when actuated.

As a device 26 is removed and the walls of the bulb 160 return to their original position and fluid is ejected from the chamber 164 through the fluid path hole 152, fluid is forced from the vascular access device through the extravascular system 28 and into the vascular system of a patient. Such fluid travel prevents or limits the likelihood that blood would travel against this flow of fluid from the vascular system of a patient into a portion of the extravascular system 28.

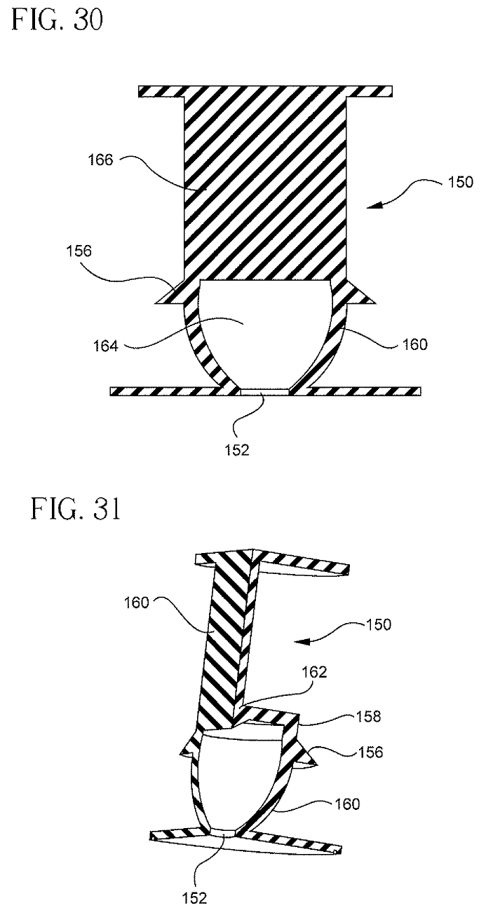

Referring now to FIG. 30, the septum 150 of FIGS. 28 and 29 is shown in cross section view at a ninety degree angle from the view of FIG. 29. The septum 150 shows the interior surface of the slit 166, the wedge 156, the internal chamber 164, the bulb 160, and the fluid path hole 152.

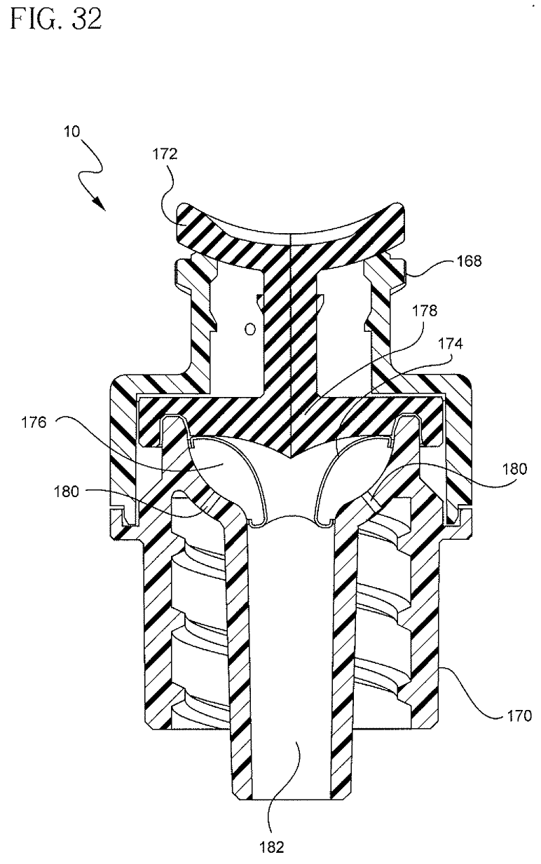

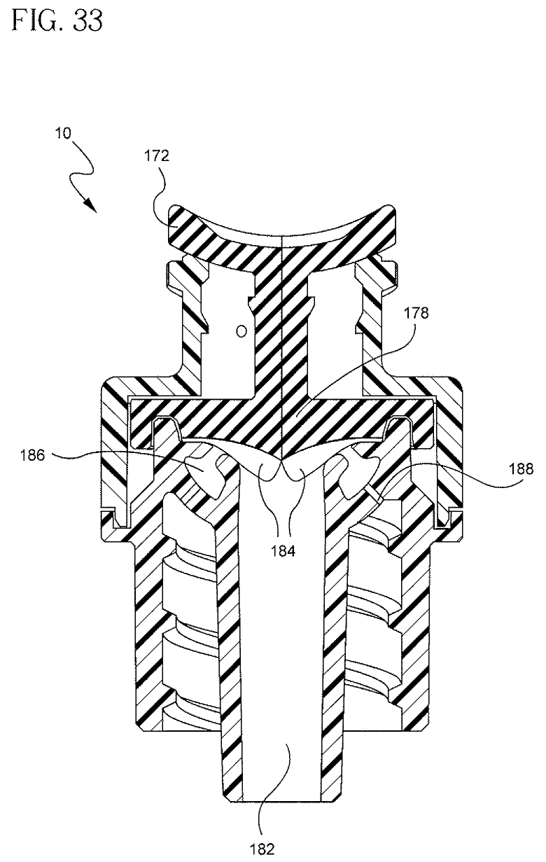

Referring now to FIG. 31, a quarter section of the septum 150 of the FIGS. 28 through 30 is shown. This quarter section shows the slit 166, the floor 162, the straight vertical containment wall 158, the wedge 156, the internal chamber 164, the thin sidewall of the bulb 160, and a section of the fluid path hole 152. As mentioned earlier, the structure of the wedge 156 and the vertical containment wall 158 forces the floor 152 of the septum 150 into its original, closed, resting position after the tip 30 of a separate device 26 is removed from the slit 166. The embodiment of FIGS. 28 through 31 thus reveals an elastomer that is a bulb 160 that expands when the access port or septum 150 of a vascular access device 10 is accessed by a separate device 26.

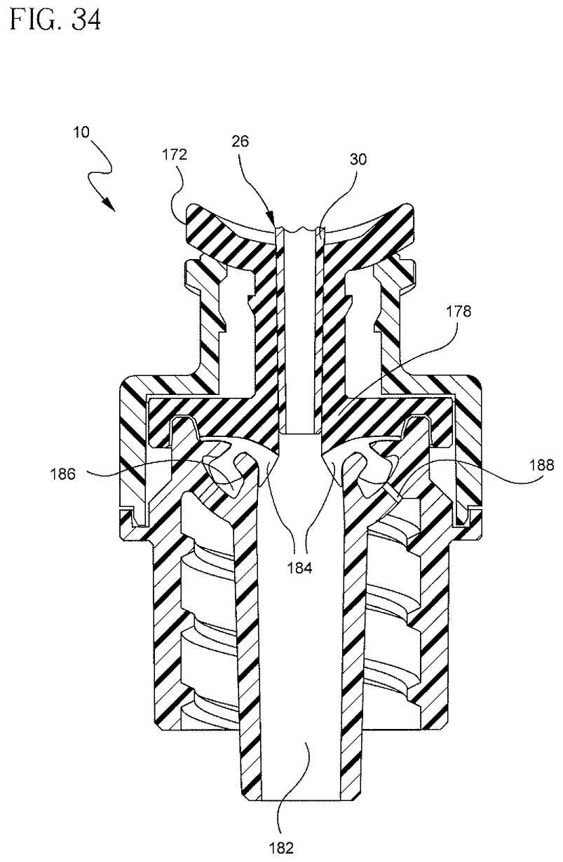

Referring now to FIG. 32, a cross section view of a vascular access device 10 shows a top housing 168 attached to a bottom housing 170. An elastomeric septum 172 resides within the top housing 168 and communicates with the wall 174 of a compression balloon or flush dome 176 such that when the floor 178 of the septum is forced downward by the tip 30 of a separate device 26, the wall 174 of the compression balloon 176 collapses causing fluid or air that is housed within the compression balloon 176 to escape through a venting hole 180. When the tip 30 of a separate device 26 is removed from the septum 172, the floor 178 of the septum 172 will return to its original position and the wall 174 of the compression balloon 176 will likewise return to its original position, erecting after the tip 30 has been removed.

Thus, the embodiment shown in FIG. 32 is a vascular access device 10 with an elastomer capable of permitting insertion and withdrawal of the tip 30 of a separate device 26 without any displacement of volume within a downstream chamber 182. Because there is no displacement of volume within the chamber 182, the blood of the vascular system of a patient is not likely to enter into the chamber 182 or any downstream chamber in communication therewith during insertion and/or removal of the tip 30.

Referring now to FIG. 33, a vascular access device 10 includes a chamber beneath the floor 178 of a septum 172 that is filled with gel, closed-cell foam, or another substance 184 and an adjacent relief cavity 186 having a venting hole 188.

Referring now to FIG. 34, the vascular access device 10 of FIG. 31 is shown with the male tip 30 of a separate device 26 inserted into the septum 172 causing the floor 178 to move downward and outward, pressing against the substance 184 and displacing the substance from the cavity beneath the floor 178 into the relief cavity 186. When the tip 30 of the separate device 26 is removed from the septum 172, the floor 178 returns to its original unactuated position shown in FIG. 33 and the substance 184 likewise moves from the relief cavity 186 into the cavity beneath the floor 178. Thus, similar to the embodiment of FIG. 32, the embodiment of FIGS. 33 and 34 reveal a structure or system similar to a compression balloon which provides little to no displacement of volume within a chamber 182 that is downstream from the insertion of the tip 30 of a separate device 26.

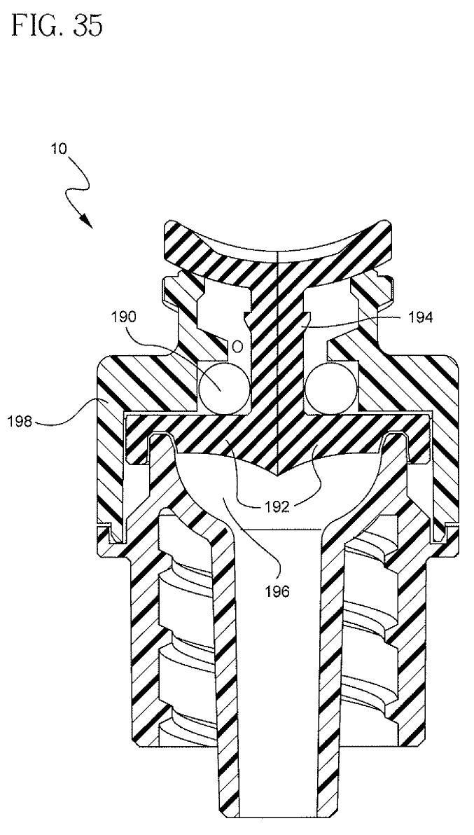

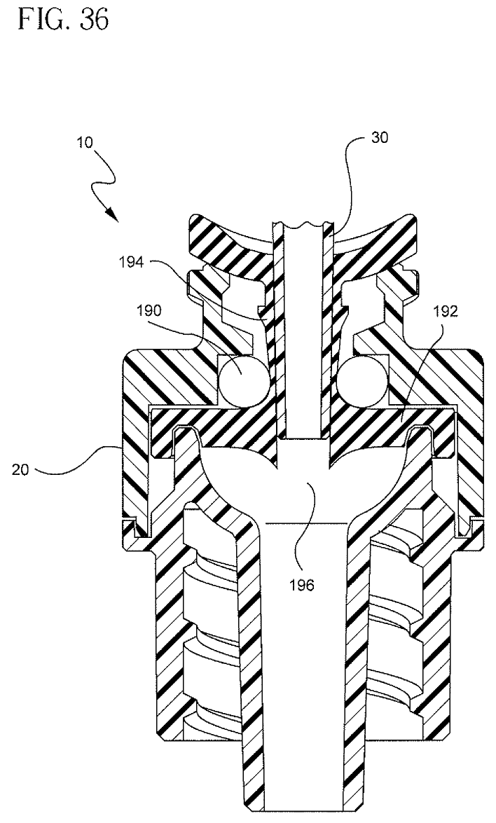

Referring now to FIGS. 35 and 36. In FIG. 35 a vascular access device 10 is illustrated prior to tip 30 insertion. FIG. 36 illustrates the device during tip 30 insertion. The vascular access device includes a radial compression spring 190 exerting force upon the floor 192 of a septum 194. Thus, the device 10 in its unactuated, resting position prior to tip 30 insertion, includes a small amount of volume within a chamber 196 beneath the floor 192 of the septum 194. After the tip 30 is inserted, as shown in FIG. 36, the tip 30 causes the septum 194 and its floor 192 to open outwards, forcing the radial spring 190 to move from a first position shown in FIG. 35 to a second position shown in FIG. 36. When the radial spring 190 moves into a second position, the floor 192 of the septum 194 raises causing an increased amount of volume within chamber 196. After the tip 30 is removed, the radial spring 190 and floor 192 return to their original position, decreasing the volume within the chamber 196.

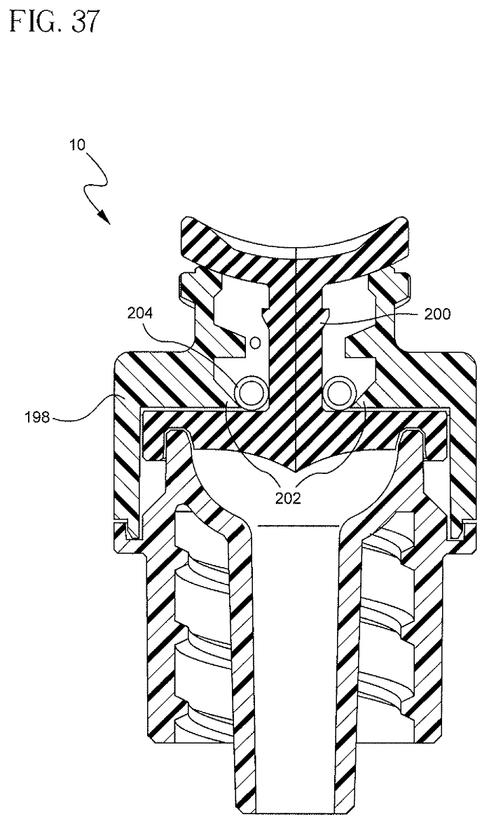

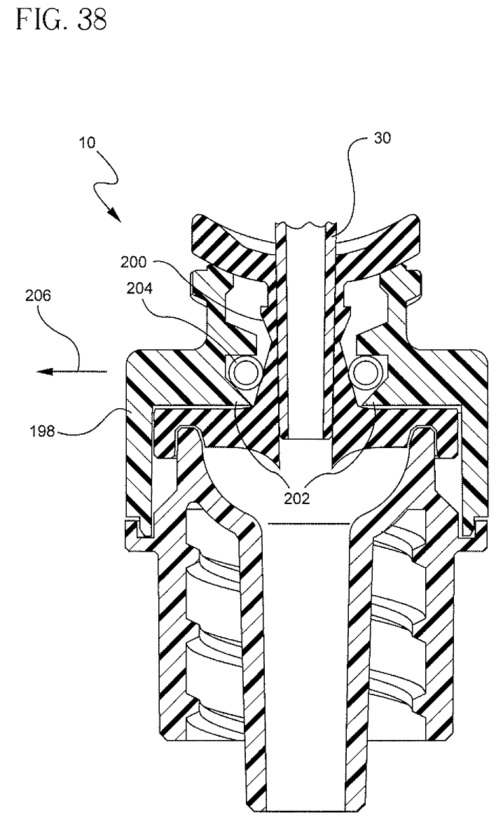

Referring now to FIG. 37, a partial cross section view of a vascular access device 10 includes a body 198 and an elastomer 200 with a ramp 202 or a relatively rigid component or section of the elastomer 200. The ramp 202 is in communication with an o-ring or compression spring 204.

Referring now to FIG. 38, the partial cross section view of the vascular access device 10 is shown after a male Luer or tip 30 has been inserted into the device 10 forcing the elastomer to travel in an outward direction 206. As the elastomer 200 moves in a direction 206, the o-ring 204 is forced up the ramp 202 with which it communicates, permitting the elastomer 200 to provide an increased amount of space or volume in a chamber adjacent or below the elastomer 200. When the increased amount of volume is created within the chamber that is adjacent the elastomer 200, the increased amount of volume will offset any decrease of volume caused by the insertion of the tip 30. The volume offset will prevent or otherwise limit blood from flowing from the vascular system of a patient into the extravascular system 28 to which the device 10 is attached as the tip 30 is removed from the device 10.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A medical device, comprising:
a vascular access device with an access port having a septum and a slit, wherein the slit is formed on the inner surface of the body of the septum, wherein the access port receives a separate access device through the slit of the septum; and
a flexible member, wherein the flexible member expands to create additional volume within the port when the port is accessed by the access device such that when the separate access device is removed from the vascular access device, fluid is not drawn into the vascular access device.

2. The medical device of claim 1, wherein access by the separate access device includes fluid infusion into the port.

3. The medical device of claim 2, wherein the flexible member is a peristaltic catheter for delivering a bolus of the fluid along the length of the peristaltic catheter.

4. The medical device of claim 2, wherein the flexible member is an elastomeric gate.

5. The medical device of claim 1, further comprising a check valve through which the fluid is infused.

6. The medical device of claim 2, wherein the flexible member is a balloon housed within a chamber.

7. The medical device of claim 1, wherein the flexible member forms a twisted fluid path that untwists as the flexible member expands.

8. The medical device of claim 1, further comprising an air pressure chamber, wherein the volume within the port is housed within a fluid chamber, and wherein the volume of the fluid chamber increases as the volume of the air pressure chamber changes.

9. The medical device of claim 1, further comprising a pressure sensitive chemical chamber, wherein the volume within the port is housed within a fluid chamber, and wherein the volume of the fluid chamber increases as the volume of the pressure sensitive chemical chamber changes.

10. The medical device of claim 1, wherein access by the separate access device includes the insertion of a mechanical structure into the port.

11. The medical device of claim 10, further comprising a strut in communication with the flexible member, wherein the flexible member expands as the strut compresses.

12. The medical device of claim 10, wherein the flexible member is a bulb that expands when the port is accessed by the device.

13. The medical device of claim 10, wherein the flexible member forms the wall of a compression balloon and expands as the compression balloon is compressed.

14. The medical device of claim 10, further comprising a radial compression spring, wherein the flexible member expands as the radial compression spring moves.

15. A method of controlling volume displacement of a chamber of a medical device, comprising:
decreasing the volume of a chamber of an extravascular system by inserting a substance having a mass into the chamber, and
increasing the volume of the chamber simultaneously and commensurately with the mass of the substance inserted into the chamber such that upon removal of the substance having a mass, fluid is not drawn into the chamber.

16. The method of claim 15, wherein the substance is a mechanical structure of a medical device.

17. The method of claim 16, wherein the mechanical structure is a tip of a syringe.

18. The method of claim 15, wherein the substance is a fluid.

19. A medical device, comprising:
a means for increasing the volume of a chamber in an extravascular system,
wherein the means for increasing the volume commensurately communicates with a means for decreasing the volume of the chamber.

20. The medical device of claim 19, wherein the means for increasing the volume is housed within a closed Luer access device.
